(19) United States
(12) Patent Application Publication
(10) Pub. No.: US 2013/0046243 A1
(43) Pub. Date: Feb. 21, 2013

(54) Holding Device for an Infusion Needle

(71) Applicant: Kuo-Cheng Wu, Kaohsiung City (TW)

(72) Inventor: Kuo-Cheng Wu, Kaohsiung City (TW)

(21) Appl. No.: 13/213,203

(22) Filed: Aug. 19, 2011

(51) Int. Cl. A61M 5/158

(52) U.S. Cl. 604/180

(57) ABSTRACT

A holding device for an infusion needle includes a cannula having a cannula body and a needle on an end of the cannula body. A wing unit includes at least one wing mounted to an outer periphery of the cannula body of the cannula. The at least one wing includes a bottom engagement face. A sticking member includes at least one adhesive tape having a fixing face and a sticking face. The fixing face of the at least one adhesive tape is engaged with the bottom engagement face of the at least one wing. The sticking face includes at least one groove, dividing the sticking face into a sticking section and a recessed section. The sticking section is other than the at least one groove. The recessed section corresponds to the at least one groove.

### BACKGROUND OF THE INVENTION

1. Field of the Invention

The present invention relates to a holding device for an infusion needle and, more particularly, to a holding device that can reliably hold an infusion needle in a predetermined infusion location to allow rapid and simple infusion.

2. Description of the Related Art

Conventional infusion needles are generally used for infusing or drawing liquids. The infusion needles should be effectively retained in place during infusion or drawing so as to finish the infusion or drawing.

FIG. 1 shows a conventional infusion needle 8 for drawing blood or injection administration on a patient. The infusion needle 8 includes a cannula 81 having a needle 82 on an end thereof When a medical worker insert the needle 82 of the cannula 81 into a predetermined location of the body of a patient, an adhesive tape 83 is used to fix the cannula 81 on the body adjacent to the predetermined location. This avoids the patient from pain resulting from repeated insertion of the infusion needle 8 while allowing repeated drawing of blood or repeated injection by the medial worker.

However, after inserting the needle 82 into the predetermined location of the patient, the medical worker still has to hold the cannula 81 with one hand and to apply the adhesive tape 83 to fix the cannula 81 with the other. It is not easy to position the cannula 81 due to swaying of the hand of the medical worker, adversely affecting the positioning accuracy of the needle 82. Furthermore, operation of both hands of the medical worker for simultaneously holding and fixing the cannula 81 is inconvenient. Further, when the medical worker intends to remove the infusion needle 8, the patient will feel pain and uncomfortable while removing the adhesive tape 83 having an adhesive side that completely sticks to the skin surface adjacent to the predetermined location.

FIG. 2 shows a one-time safe butterfly needle 9 including a protective cannula 91 slideably receiving a sliding member 92 having a push section 921 on an upper edge thereof. A needle is mounted to a front end of the sliding member 92. Two wings 93 are mounted to a side of the protective cannula 91 and each includes a sticking portion 94. In use, a medical worker pushes the sliding member 92 by the push section 921 such that the needle of the sliding member 92 slides out of the protective cannula 91 into a predetermined point of the body of a patient. Then, a peel paper 941 is peeled off from each sticking portion 94, and the sticking portion 94 is turned over about a connection between the sticking portion 94 and the wing 93 and sticks to the skin of the body adjacent to the predetermined point. Thus, the medical worker can perform repeated drawing of blood or repeated injection through use of the safe butterfly needle 9. An example of such an infusion needle 9 is disclosed in Taiwan Patent Publication No. 200526291.

However, after inserting the needle of the safe butterfly needle 9 into the predetermined location of the patient, the medical worker still has to hold the protective cannula 91 or the wings 93 with one hand and to peel off the peel papers 941 for sticking of the sticking portions 94. The whole operation is complicated and time-consuming, causing inconvenience to the medical worker if the safe butterfly needle 9 is used in a medical environment requiring efficiency. Furthermore, it is not easy to position the safe butterfly needle 9 due to swaying of the hand of the medical worker while peeling the peel papers 941, adversely affecting the positioning accuracy of the needle. Furthermore, when the medical worker intends to remove the sticking portions 94 from the predetermined location of the patient, the patient will feel pain and uncomfortable. Improvement is, thus, required.

### SUMMARY OF THE INVENTION

An objective of the present invention is to provide a holding device for an infusion needle that can be rapidly and simply retained in place by sticking after a needle of the infusion needle is inserted into a predetermined location of a patient.

Another objective of the present invention is to provide a holding device for an infusion needle that can reliably retain the needle during sticking of the infusion needle, such that the needle can be inserted into the predetermined location with enhanced accuracy.

A further objective of the present invention is to provide a holding device for an infusion needle that can effectively ease the pain of the patient while removing the infusion needle.

The present invention fulfills the above objectives by providing, a holding device for an infusion needle including a cannula having a cannula body and a needle on an end of the cannula body. A wing unit includes at least one wing mounted to an outer periphery of the cannula body of the cannula. The at least one wing includes a bottom engagement face. A sticking member includes at least one adhesive tape having a fixing face and a sticking face. The fixing face of the at least one adhesive tape is engaged with the bottom engagement face of the at least one wing. The sticking face includes at least one groove, dividing the sticking face into a sticking section and a recessed section. The sticking section is other than the at least one groove. The recessed section corresponds to the at least one groove.

In a form, the cannula is connected to an infusion assembly. The infusion assembly includes a hose having a first end coupled to the cannula body of the cannula and a second end. A coupling member is provided on the second end of the hose. The coupling member includes an opening, and a plug is removably engaged in the opening. The infusion assembly can further include a flow control member connected to the hose. The flow control member is adapted to control a flow rate of a liquid flowing through the hose.

In a form, the sticking member includes a plurality of spaced grooves, forming a plurality of spaced recessed sections and a plurality of sticking sections in the sticking face of the sticking member. Each of the plurality of sticking sections is located between two adjacent recessed sections.

In a form, the at least one adhesive tape includes an outer edge spaced from an outer edge of the at least one wing. The at least one wing has an extension relative to the at least one adhesive tape.

In a form, the bottom engagement face of the wing unit includes a plurality of engagement grooves. The fixing face of the sticking member includes a plurality of protrusions. Each of the plurality of protrusions is engaged in one of the plurality of engagement grooves.

The present invention will become clearer in light of the following detailed description of illustrative embodiments of this invention described in connection with the drawings.

### BRIEF DESCRIPTION OF THE DRAWINGS

The illustrative embodiments may best be described by reference to the accompanying drawings where:

FIG. 1 shows a perspective view of a conventional infusion needle.

FIG. 2 shows a perspective view of a conventional safe butterfly needle.

FIG. 3 shows an exploded, perspective view of a holding device for an infusion needle of an embodiment according to the present invention.

FIG. 4 shows a cross sectional view taken along section line 4-4 of the holding device of FIG. 3 after assembly.

FIG. 5 shows a cross sectional view of the holding device for an infusion needle of FIG. 3, wherein the holding device sticks to a skin of a patient.

FIG. 6 shows a cross sectional view similar to FIG. 5, illustrating removal of the holding device.

FIG. 7 shows an exploded, perspective view of a holding device for an infusion needle of another embodiment according to the present invention.

FIG. 8 shows a cross sectional view taken along section line 8-8 of the holding device of FIG. 7 after assembly.

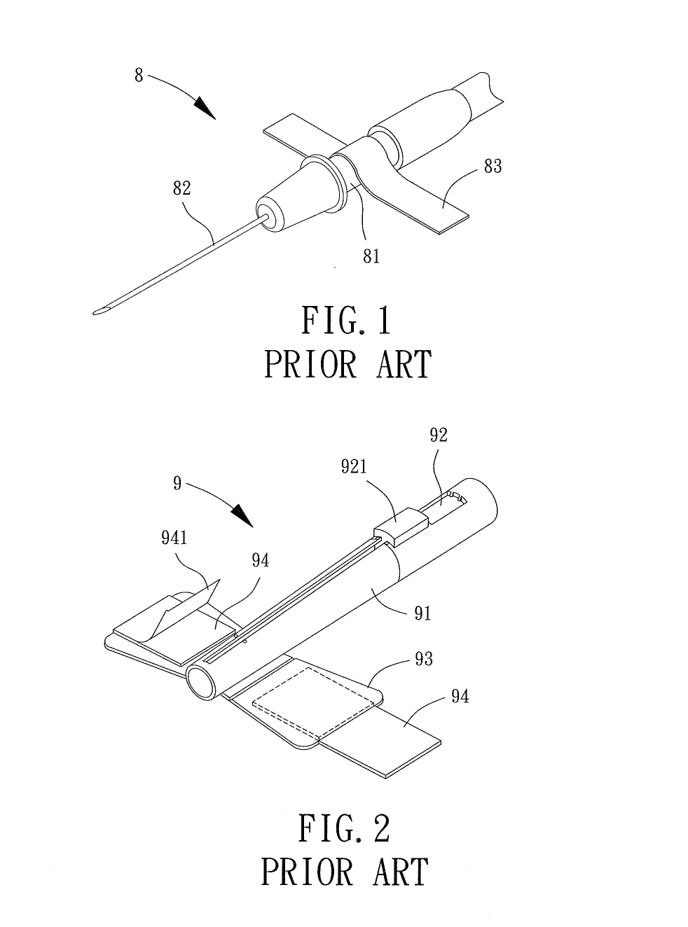

All figures are drawn for ease of explanation of the basic teachings of the present invention only; the extensions of the figures with respect to number, position, relationship, and dimensions of the parts to form the preferred embodiments will be explained or will be within the skill of the art after the following teachings of the present invention have been read and understood. Further, the exact dimensions and dimensional proportions to conform to specific force, weight, strength, and similar requirements will likewise be within the skill of the art after the following teachings of the present invention have been read and understood.

Where used in the various figures of the drawings, the same numerals designate the same or similar parts. Furthermore, when the terms “first”, “second”, “outer”, “side”, “end”, “portion”, “section”, “length”, and similar terms are used herein, it should be understood that these terms have reference only to the structure shown in the drawings as it would appear to a person viewing the drawings and are utilized only to facilitate describing the invention.

### DETAILED DESCRIPTION OF THE INVENTION

A holding device for an infusion needle according to the present invention can be used for various infusions or drawing of liquids. For ease of explanation, it is assumed that the holding device for an infusion needle is used in drawing blood or injection administration.

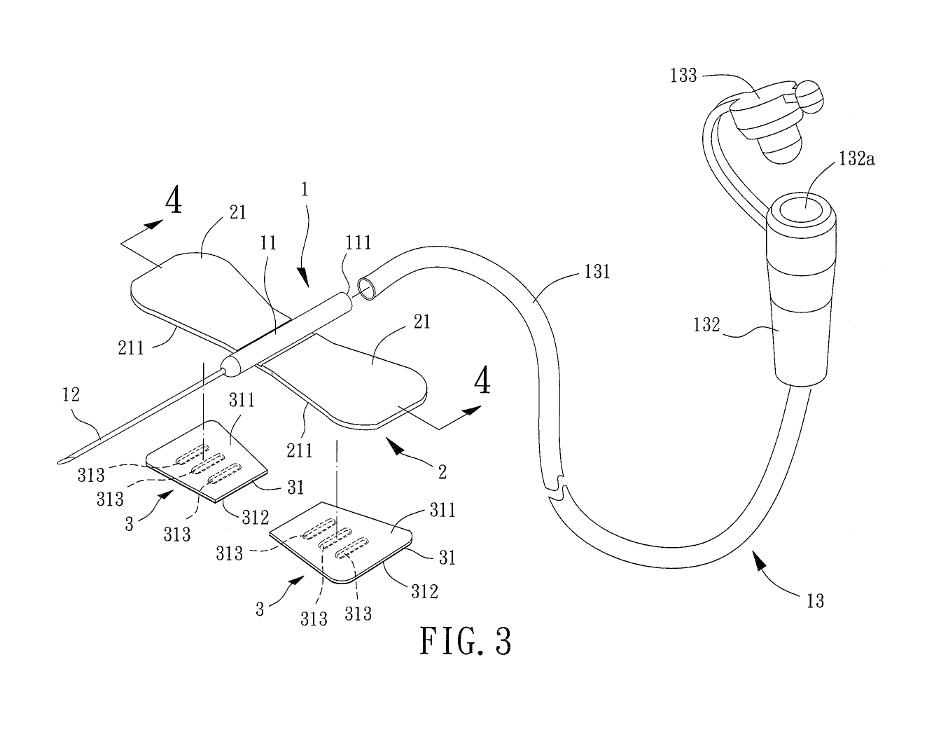

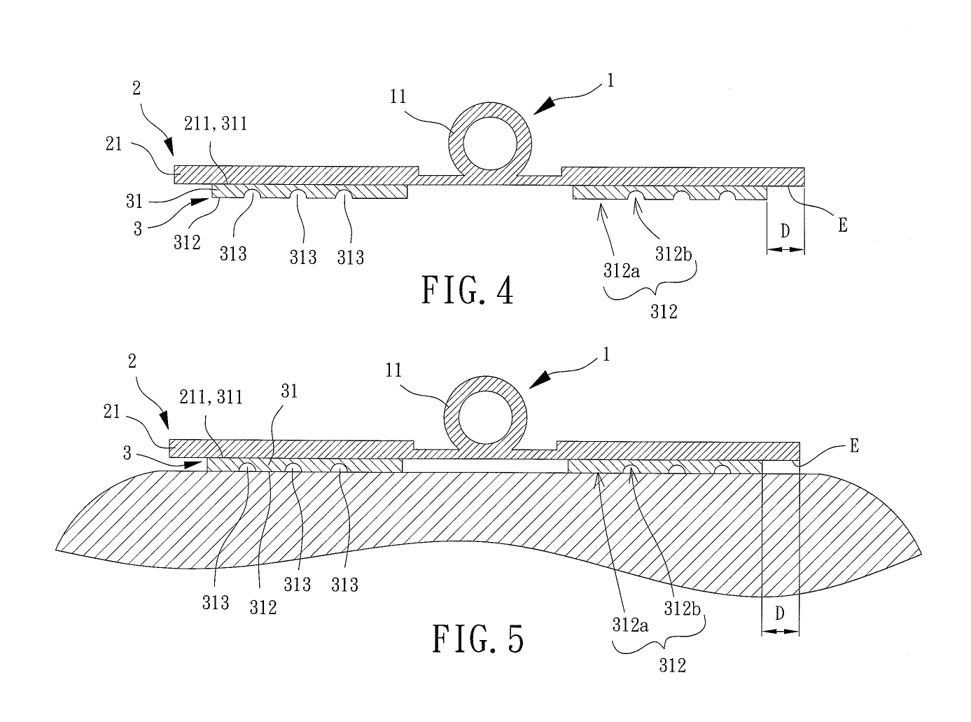

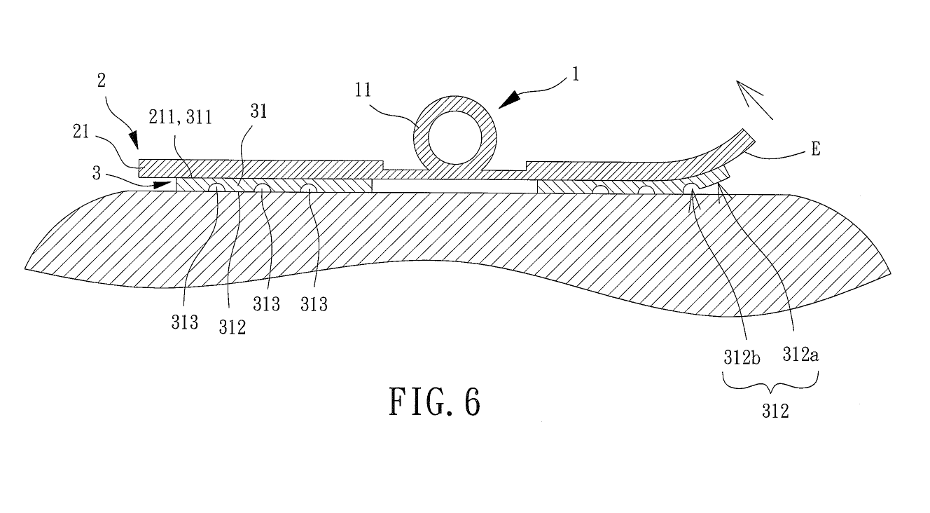

With reference to FIGS. 3 and 4, a holding device for infusion of an embodiment according to the present invention includes a cannula 1, a wing unit 2 and a sticking member 3. The cannula 1 is in the form of a hollow tube for delivery of administration, blood or any suitable liquid. The wing unit 2 engages with the cannula 1. The sticking member 3 engages with the wing unit 2 to permit a user to fix the cannula 1.

The cannula 1 includes a cannula body 11 and a needle 12. The cannula body 11 includes an engagement portion 111 on an end thereof for engagement with various infusion assemblies 13 according to different needs so that the holding device for an infusion needle according the present invention can provide other functions (which will be described in detail hereinafter). The engagement portion 111 and the infusion assembly 13 can be engaged with each other by male/female coupling, bonding, screwing, or any other suitable provision. The needle 12 is located on the other end of the cannula body 11 (away from the engagement portion 111 in the embodiment shown). The needle 12 is in communication with the cannula body 11. Thus, after the needle 12 is inserted into a predetermined location of a patient, a medical worker can proceed with blood drawing or injection administration through the cannula body 11.

The wing unit 2 can be comprised of one or more wings having a geometric shape suitable to be gripped by the medical worker. In this embodiment, the wing unit 2 is comprised of two wings 21 generally in the form of a pair of butterfly wings to allow easy gripping by the medical worker. Each wing 21 has an end mounted to an outer periphery of the cannula body 11 of the cannula 1. Each wing 21 further includes a bottom engagement face 211 for engagement with the sticking member 3.

The sticking member 3 can be comprised of one or more adhesive tapes. As an example, the sticking member 3 can be a single adhesive tape engaged with the wing unit 2 comprised of a single wing. As another example, the sticking member 3 can be a single adhesive tape having a large area engaged with a plurality of wings of the wing unit 2. As a further example, the sticking member 3 can be comprised of a plurality of adhesive tapes respectively engaged with a plurality of wings 21 of the wing unit 2. The shape of the sticking member 3 preferably corresponds to that of the wings 21 of the wing unit 2. In this embodiment, the sticking member 31 includes two adhesive tapes 31 each having a fixing face 311 and a sticking face 312. The fixing face 311 of each adhesive tape 31 is in intimate contact with the bottom engagement face 211 of the wing 21 so that the adhesive tape 31 is inseparable from the wing 21 unless subject to destructive forcible detachment. The sticking face 312 includes at least one groove 313. With reference to FIG. 4, the sticking face 312 of each adhesive tape 31 is divided into a sticking section 312a and a recessed section 312b. The sticking section 312a is the section other than the groove 313. The recessed section 312b corresponds to the groove 313.

The wing unit 2 can be integrally formed with the cannula 1 by injection molding. Then, the sticking member 3 is integrally formed with the wing unit 2 by a second injection molding. Thus, more reliable engagement is provided between the cannula 1, the wing unit 2 and the sticking member 3 while enhancing assembling convenience.

With reference to FIGS. 3 and 5, in use of the holding device for an infusion needle according to the present invention, a medical worker can overlap and grip the wings 21 of the wing unit 2 so as to insert the needle 12 of the cannula 1 into a predetermined location (such as on an arm) of a patient. Then, the medical worker can flatten the wings 21 of the wing unit 2 on the skin adjacent to the predetermined location. The sticking faces 312 of the adhesive tapes 31 of the sticking member 3 stick to the skin of the patient, reliably fixing the cannula 1 to the predetermined location. This allows convenient repeated blood drawing or repeated injection by the medical worker and prevents the patient from pain due to repeated insertion of the needle 12 of the cannula 1.

The main feature of the holding device for an infusion needle according to the present invention is that the fixing faces 311 of the sticking member 3 are in intimate contact with the bottom engagement faces 211 of the wing unit 2 such that the adhesive tapes 31 and the wings 21 are in inseparable, fixed, engagement relation. Thus, after the medical worker inserts the needle 12 of the cannula 1 into the predetermined location of the patient and manually presses the wing unit 2 to flatten the wings 21 on the predetermined location, the medical worker can assure direct sticking of the sticking faces 312 of the sticking member 3 to the skin of the patient without turning over the sticking member 3. In overall, the sticking/fixing way of the holding device for an infusion needle according to the present invention is simple so that the medical worker can rapidly and accurately finish the sticking operation while avoiding inaccurate positioning of the needle 12 due to swaying of the hand of the medical worker during the sticking operation.

Of more importance, if the medical worker intends to remove the holding device for an infusion needle according to the present invention from the predetermined location of the patient by peeling the sticking member 3 from the skin of the patient, due to provision of the groove 313 on the sticking face 312 (and, thus, forms the sticking section 312a and the recessed section 312b), the recessed section 312b can effectively ease the pain of the patient during peeling of the sticking member 3, allowing the medical worker to more easily peel the sticking member 3. Furthermore, due to provision of the sticking section 312a on the sticking face 312 other than the recessed section 312b, the sticking/fixing effect of the holding device for an infusion needle according to the present invention is not adversely affected.

Based on the technical concept of the holding device for an infusion needle according to the present invention, the holding device can include other features to provide more functions.

With reference to FIGS. 3 and 4, when the fixing face 311 of each adhesive tape 31 intimately sticks to the bottom engagement faces 211 of the wings 21, an outer edge of each adhesive tape 31 is spaced from an associated wing 21 such that each wing 21 has an extension E relative to the adhesive tape 31. By such an arrangement, when the medical worker holds the extension E by his or her hand, the needle 12 of the cannula 1 can be easily inserted into the predetermined location of the patient, providing enhanced convenience in use.

With reference to FIGS. 3 and 4, the sticking member 3 can include a plurality of grooves 313. The grooves 313 are spaced from each other such that the sticking face 312 of the sticking member 3 includes a plurality of spaced recessed sections 312b. A sticking section 312a is located between two adjacent recessed sections 312b. By such an arrangement, the recessed sections 312b can more effectively ease the pain of the patient while peeling the sticking member 3 from the skin of the patient. Furthermore, the sticking section 312a between two adjacent recessed sections 312b can securely engage with the area adjacent to the predetermined point of the patient.

With reference to FIG. 3, the infusion assembly 13 includes a hose 131 having an end engaged with the engagement portion 111. A hollow coupling member 132 is provided on the other end of the hose 131 and is in communication with the hose 131. An end of the coupling member 132 distal to the hose 131 includes an opening 132a and a plug 133 removably engaged in the opening 132a. Since the hose 131 of the infusion assembly 13 has a predetermined length and flexibility, after the cannula body 11 and the needle 12 of the cannula 1 are connected to the body of the patient, the hose 131 can extend to a site permitting the medical worker to easily proceed with blood drawing or injection administration, providing enhanced convenient use. Furthermore, after blood drawing or injection administration, the plug 133 can seal the opening 132a of the coupling member 132, avoiding entrance of alien objects, such as dust, into the hose 131 or the cannula body 11, effectively increasing medial safety and quality.

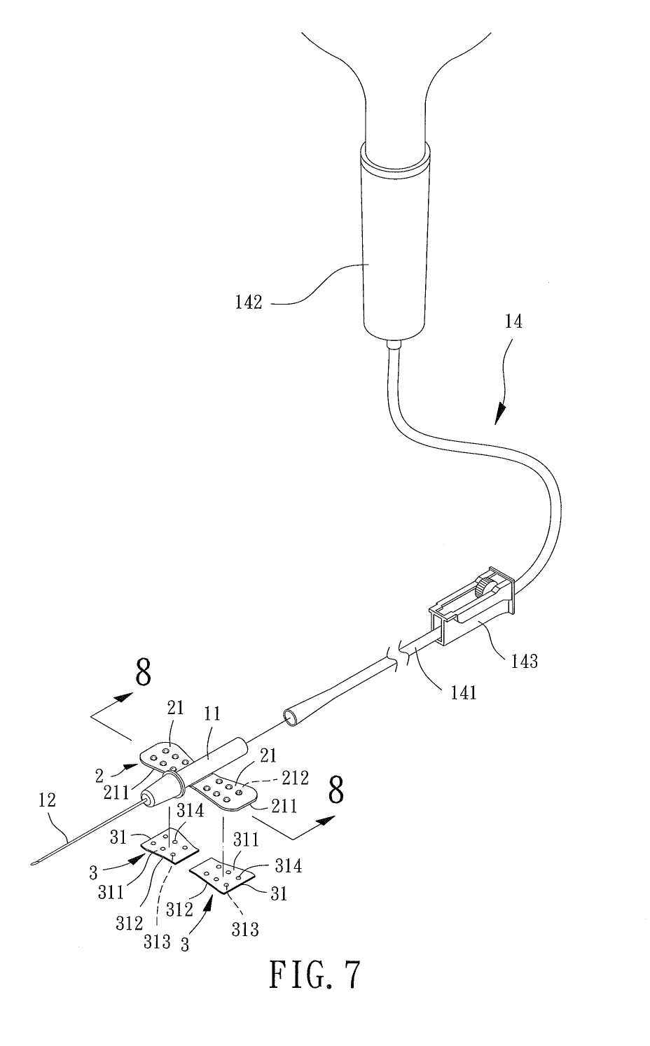

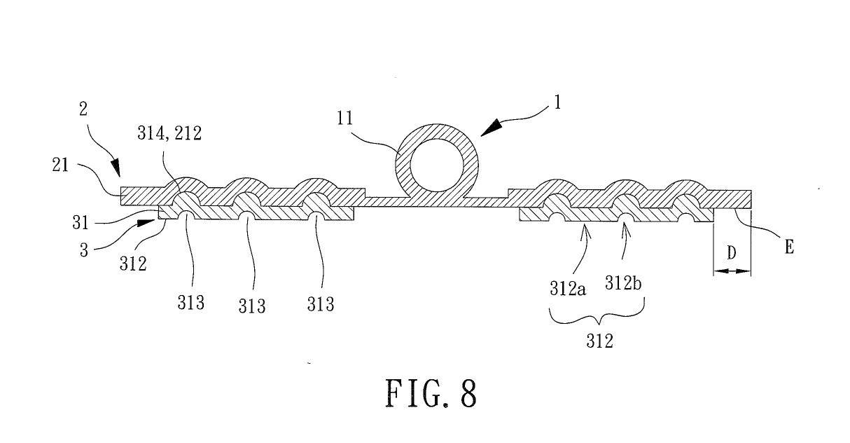

FIGS. 7 and 8 show another embodiment of the infusion assembly 14. The infusion assembly 14 includes a hose 141 having an end coupled to the engagement portion 111 of the cannula body 11. A coupling member 142 is provided on the other end of the hose 141. The coupling member 142 is hollow and in communication with the hose 141. The infusion assembly 14 further includes a flow control member 143 connected to the hose 141 for controlling the flow rate of the liquid flowing through the hose 141. The coupling member 142 of the infusion assembly 14 can be connected to an intravenous bottle. Through use of the flow control member 143 to control the flow rate of administration passing through the hose 141, the holding device for an infusion needle according to the present invention provides better utility while being used in the medical field.

With reference to FIGS. 7 and 8, the bottom engagement face 211 of the wing unit 2 of the holding device for an infusion needle according to the present invention can further include a plurality of engagement grooves 212. The fixing face 311 of the sticking member 3 includes a plurality of protrusions 314 corresponding to the engagement grooves 212. Each protrusion 314 is engaged in one of the engagement grooves 212, effectively increasing the engagement area between the bottom engagement faces 211 of the wing unit 2 and the fixing face 311 of the sticking member 3 and, hence, effectively enhancing engagement reliability between the unit 2 and the sticking member 3.

In view of the foregoing, the holding device for an infusion needle according to the present invention provides intimate contact between the fixing faces 311 of the sticking member 3 and the bottom engagement face 211 of the wing unit 2 such that the adhesive tape 31 and the wings 21 are in inseparable, fixed, engagement relation. Thus, after the needle 12 is inserted into the predetermined location of the patient, the medical worker does not have to turn over the adhesive tape 31, more rapidly and easily completing the sticking operation. Enhanced convenient use is, thus, provided.

Furthermore, through mutual engagement between the sticking member 3 and the wing unit 2, the holding device for an infusion needle according to the present invention permits the user to reliably position the holding device during the sticking operation. Thus, the needle 12 can be more accurately inserted into the predetermined location on the patient, providing enhanced positioning accuracy.

Further, by providing the sticking faces 312 with the sticking sections 312a and the recessed sections 312b, the holding device for an infusion needle according to the present invention can ease the pain of the patient while the user (medical worker) is removing the holding device, providing enhanced medical quality.

Thus since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

What is claimed is:

1. A holding device for an infusion needle comprising:
a cannula including a cannula body and a needle on an end of the cannula body;
a wing unit including at least one wing, with said at least one wing mounted to an outer periphery of the cannula body of the cannula, with said at least one wing including a bottom engagement face; and
a sticking member including at least one adhesive tape, with said at least one adhesive tape including a fixing face and a sticking face, with the fixing face of said at least one adhesive tape engaged with the bottom engagement face of said at least one wing, with the sticking face including at least one groove, with the sticking face divided into a sticking section and a recessed section, with the sticking section being other than said at least one groove, with the recessed section corresponding to said at least one groove.

2. The holding device for an infusion needle as claimed in claim 1, with the cannula connected to an infusion assembly, with the infusion assembly including a hose having a first end coupled to the cannula body of the cannula and a second end, with a coupling member provided on the second end of the hose.

3. The holding device for an infusion needle as claimed in claim 2, with the coupling member including an opening, with a plug removably engaged in the opening.

4. The holding device for an infusion needle as claimed in claim 2, with the infusion assembly further including a flow control member connected to the hose, with the flow control member adapted to control a flow rate of a liquid flowing through the hose.

5. The holding device for an infusion needle as claimed in claim 1, with said at least one groove of the sticking member including a plurality of spaced grooves, forming a plurality of spaced recessed sections and a plurality of sticking sections in the sticking face of the sticking member, with each of the plurality of sticking sections located between two adjacent recessed sections.

6. The holding device for an infusion needle as claimed in claim 1, with said at least one adhesive tape including an outer edge spaced from an outer edge of said at least one wing, with said at least one wing having an extension relative to said at least one adhesive tape.

7. The holding device for an infusion needle as claimed in claim 1, with the bottom engagement face of the wing unit including a plurality of engagement grooves, with the fixing face of the sticking member including a plurality of protrusions, with each of the plurality of protrusions engaged in one of the plurality of engagement grooves.

8. The holding device for an infusion needle as claimed in claim 1, with the wing unit comprised of two wings in the form of a pair of butterfly wings.
